(19) United States
(12) Patent Application Publication
(10) Pub. No.: US 2008/0125664 A1
(43) Pub. Date: May 29, 2008

(54) Pulse wave detection apparatus

(75) Inventors: Kazuhiro Sakai, Toyoake-city (JP); Katsuyoshi Nishii, Okazaki-city (JP)

Correspondence Address: POSZ LAW GROUP, PLC, 12040 SOUTH LAKES DRIVE, SUITE 101, RESTON, VA, 20191, US

(73) Assignee: DENSO CORPORATION, Kariya-city (JP)

(21) Appl. No.: 11/979,969

(22) Filed: Nov. 13, 2007

(30) Foreign Application Priority Data
Nov. 27, 2006 (JP) 2006-318710

(51) Int. Cl. A61B 5/02

(52) U.S. Cl. 600/500

(57) ABSTRACT

An optical pulse wave detection apparatus includes a light emitting element, a light receiving element, a first control unit, a second control unit, and a pulse wave detection unit. The first control unit obtains a first signal by causing the light emitting element to emit light of a first light quantity, and by causing the light receiving element to receive reflected light, which is reflected by a living organism, of the light of the first light quantity. The second control unit obtains a second signal by causing the light emitting element to emit the light of a second light quantity that is smaller than the first light quantity, and by causing the light receiving element to receive the reflected light of the light of the second light quantity. The pulse wave detection unit detects a pulse wave of the living organism based on the first and second signals.

### CROSS REFERENCE TO RELATED APPLICATION

This application is based on and incorporates herein by reference Japanese Patent Application No. 2006-318710 filed on Nov. 27, 2006.

### BACKGROUND OF THE INVENTION

1. Field of the Invention

The present invention relates to a pulse wave detection apparatus that detects a physiological pulse wave using a light emitting element and a light receiving element.

2. Description of Related Art

Recently, a portable equipment, such as a pedometer, a calorie consumption meter, is used for health management. Also, an equipment for monitoring heart rate during a daily life and an physical exercise (e.g., jogging) is effective for evaluation of a physical exercise amount. For example, there has been used an electrocardiographic method for measuring an action potential at a chest, which is generated along with a heart beat. Also, there has been used an optical pulse wave sensor that uses light absorption property by blood components.

The optical pulse wave sensor has a light emitting element and a light receiving element. The optical pulse wave sensor uses the light emitting element to apply light to a human body, and receives a reflected light by the light receiving element. The optical pulse wave sensor detects a pulse wave in accordance with a change of a light receiving quantity that is received. Because the sensor can be attached to a human body (e.g., finger, arm, temple of face) for a convenient measurement, the sensor may become more popular in the future (see WO 97/37588 corresponding to U.S. Pat. No. 6,241,684).

However, when the above optical pulse wave sensor is used, the difficulties below may occur, and the improvement of the sensor is necessary.

As shown in FIG. 19, in general, a peak position of an electrocardiographic waveform synchronizes with a peak position of a pulse waveform. Here, each amplitude of the electrocardiographic waveform and the pulse waveform is the largest at the corresponding peak position. Thus, the heart rate corresponds to a pulse rate. Each of the heart rate and the pulse rate is computed by dividing 60 by a peak-to-peak interval (unit of second) between the peaks of the amplitude of a corresponding one of the electrocardiographic waveform and the pulse waveform.

However, during a daily life or a physical exercise, when the optical pulse wave sensor is used outside, noise due to disturbance light disadvantageously occurs. Specifically, when disturbance light (e.g., sun light) is incident on the light receiving element, a peak of a large amplitude may be generated regardless of the heart beat because of the influence of the disturbance light. In the above case, an actual heart rate does not correspond to a pulse rate that is detected by the optical pulse wave sensor. In other words, a pulse wave component to be detected is hidden by the disturbance light, and therefore, disadvantageously, the pulse rate is not accurately detected.

Also, when the disturbance light is incident on the element, for example, an amplitude of the obtained signal may become so large that the amplitude ranges over an input voltage range. In the above case, because the detectable amplitude is limited by an upper limit or a lower limit of the input voltage range, data itself may not be accurately obtained.

### SUMMARY OF THE INVENTION

The present invention is made in view of the above disadvantages. Thus, it is an objective of the present invention to address at least one of the above disadvantages.

To achieve the objective of the present invention, there is provided an optical pulse wave detection apparatus, which includes a light emitting element, a light receiving element, a first control unit, a second control unit, and a pulse wave detection unit. The light emitting element emits light to a living organism. The light receiving element receives a reflected light of the light that is reflected by the living organism. The first control unit obtains a first signal by causing the light emitting element to emit the light of a first light quantity, and by causing the light receiving element to receive the reflected light of the light of the first light quantity. The second control unit obtains a second signal by causing the light emitting element to emit the light of a second light quantity that is smaller than the first light quantity, and by causing the light receiving element to receive the reflected light of the light of the second light quantity. The pulse wave detection unit detects a pulse wave of the living organism based on the first signal and the second signal.

To achieve the objective of the present invention, there is also provided an article manufacture, which includes a computer readable medium readable by a computer, and which includes program instructions carried by the computer readable medium for causing the computer to serve as the first control unit, the second control unit, and the pulse wave detection unit of the above optical pulse wave detection apparatus.

To achieve the objective of the present invention, there is also provided an optical pulse wave detection apparatus, which includes a light emitting element, a light receiving element, a signal control unit, and a pulse wave detection unit. The light emitting element emits light to a living organism. The light receiving element receives a reflected light of the light that is reflected by the living organism. The signal control unit causes the light emitting element to emit the light to the living organism, the signal control unit obtaining a plurality of signals from the light receiving element that receives the reflected light of the light emitted by light emitting element, each of the plurality of signals being obtained at a timing different from each other. The pulse wave detection unit detects single sampling data based on the plurality of signals, the single sampling data being used for detecting a pulse wave.

To achieve the objective of the present invention, there is also provided an article manufacture, which includes a computer readable medium readable by a computer, and which includes program instructions carried by the computer readable medium for causing the computer to serve as the signal control unit and the pulse wave detection unit in the above optical pulse wave detection apparatus.

### BRIEF DESCRIPTION OF THE DRAWINGS

The invention, together with additional objectives, features and advantages thereof, will be best understood from the following description, the appended claims and the accompanying drawings in which:

FIG. 1 is a explanatory diagram showing a general structure of a pulse wave detection apparatus according to a first embodiment of the present invention;

FIG. 2 is a explanatory diagram showing a state of using a pulse wave sensor;

FIG. 3 is a explanatory diagram showing a frequency analysis result of a pulse wave signal;

FIG. 4 is a explanatory diagram showing a normal pulse wave signal and a pulse wave signal influenced by disturbance light;

FIG. 5 is a flow chart showing a main routine of a control process according to the first embodiment;

FIG. 6 is a timing chart of the control process according to the first embodiment;

FIG. 7 is a flow chart showing a pulse rate calculation process according to the first embodiment;

FIG. 8 is a flow chart showing another pulse rate calculation process according to the first embodiment;

FIG. 9 is an explanatory diagram showing a frequency analysis result of the another pulse rate calculation process;

FIG. 10 is a flow chart showing further another pulse rate calculation process according to the first embodiment;

FIG. 11 is an explanatory diagram showing a frequency analysis result of the further another pulse rate calculation process;

FIG. 12 is an explanatory diagram separately showing a pulse wave sensor according to a second embodiment;

FIG. 13 is an explanatory diagram showing a state where first and second LEDs emit light according to the second embodiment;

FIG. 14 is an explanatory diagram showing another state where the first and second LEDs emit in another manner light according to the second embodiment;

FIG. 15 is a flow chart showing a control process according to a third embodiment;

FIG. 16 is a timing chart of the control process according to the third embodiment;

FIG. 17 is a timing chart of another control process according to the third embodiment;

FIG. 18 is a timing chart of a control process according to a fourth embodiment; and

FIG. 19 is an explanatory diagram showing a conventional art.

### DETAILED DESCRIPTION OF PREFERRED EMBODIMENTS

Embodiments of the present invention are described below referring to accompanying drawings.

### First Embodiment

a) Firstly, a structure and an operation of a pulse wave detection apparatus of the first embodiment are described.

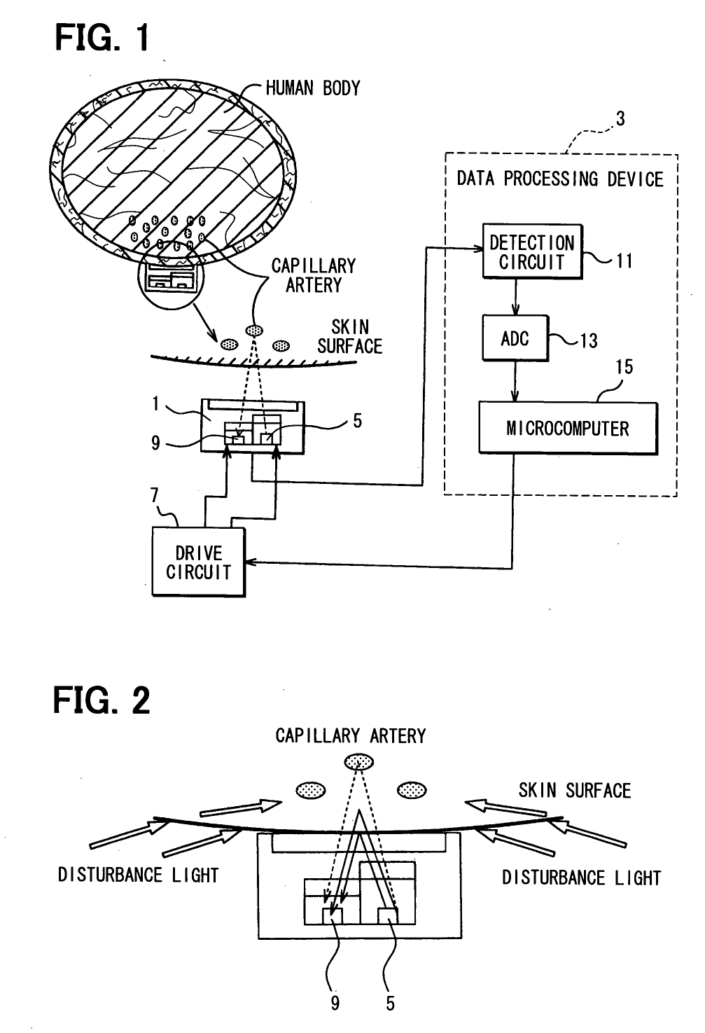

The pulse wave detection apparatus of the present embodiment measures a pulse wave of a human body to calculate a pulse rate. As shown in FIG. 1, the pulse wave detection apparatus includes a pulse wave sensor 1 and a data processing device 3. Here, the pulse wave sensor 1 is attached to the human body (e.g., arm) for use, and the data processing device 3 measures the pulse wave based on the detection result by the pulse wave sensor 1 to calculate the pulse rate.

The pulse wave sensor 1 is an optical reflection sensor that includes a light emitting diode (LED) 5, a drive circuit 7 that drives the LED 5, and a photodiode (PD) 9. The LED 5 serves as a light emitting element and the PD 9 serves as a light receiving element.

The data processing device 3 includes a detection circuit 11, an AD converter (ADC) 13, and a microcomputer 15. The microcomputer 15 stores a program for calculating the pulse rate by measuring the pulse wave signal through controlling a light quantity of the light emitted by the LED 5.

In the pulse wave detection apparatus, when light is applied to a human body from the LED 5 of the pulse wave sensor 1, some of the light hits a capillary artery that passes through the human body, and part of the light that hits the artery is absorbed by hemoglobin in blood flowing through the capillary artery. However, the rest of the light is reflected by the capillary artery to scatter, and therefore part of the reflected light is incident on the PD 9.

At the time, pulsation of the blood fluctuates the hemoglobin amount in the capillary artery. Therefore, the amount of light absorbed by the hemoglobin fluctuate. As a result, a light receiving quantity (i.e., a quantity of the light reflected by the capillary artery and then received and detected by the PD 9) changes, and therefore the change of the light receiving quantity is outputted as pulse wave information (e.g., voltage signal, electric signal) to the data processing device 3.

The detection circuit 11 of the data processing device 3 amplifies the electric signal from the PD 9, and outputs the amplified signal to the ADC 13. The ADC 13 converts the amplified analog signal into a digital signal, and inputs the digital signal to the microcomputer 15. The microcomputer 15 temporally stores the digital signal (i.e., data), and performs a calculation process for calculating the pulse rate based on the digital signal (data) by using the stored program.

b) Next, principle of the pulse wave detection according to the present embodiment is described.

As shown in FIG. 2, part of the light applied from the LED 5 to the human body enters a skin, and then hits the capillary artery to be reflected toward the PD 9. Then, the reflected light is detected as a signal (pulse wave signal) indicative of the pulse wave. Also, another part of the light is reflected by a skin surface, or is not absorbed but is reflected by the tissues of something other than the capillary artery. In general, the disturbance light travels through a gap in the sensor or enters the skin specially at an outdoor, and then, the disturbance light is superimposed on the light applied by the LED 5 and is detected by the PD 9. Thus, the waveform of the pulse wave signal detected by the PD is widely disturbed.

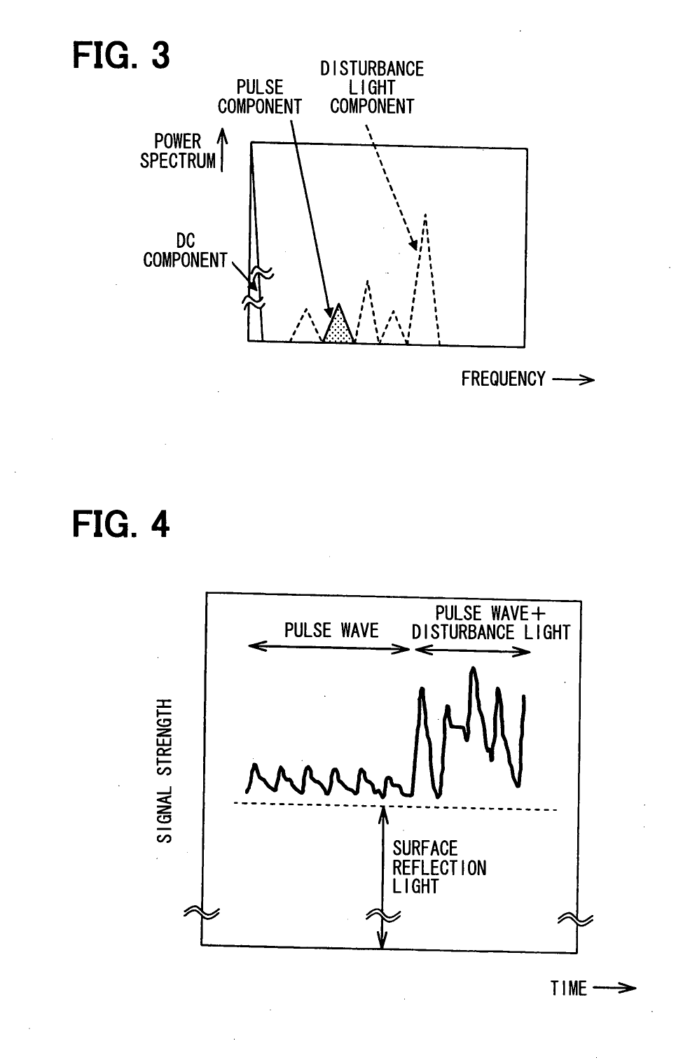

The above phenomenon is discussed based on a frequency domain that is obtainable from the pulse wave signal through a frequency analysis. As shown in FIG. 3, the pulse wave signal measure by the PD 9 includes, typically, a pulse component, a disturbance light component, and a direct-current component (DC component). Here, the pulse component synchronizes with the heart beat, and is used for obtaining the pulse rate. The disturbance light component is generated by the disturbance light, and thereby in the outside measurement, the disturbance light component may be generated significantly such that it becomes difficult to distinguish the pulse component from the disturbance light component. That is, the pulse component may be hidden by the disturbance light component in the pulse wave signal measured outside. Therefore, in order to accurately detect the pulse component, the disturbance light component is required to be eliminated. It is noted that the direct-current component can be cut by the detection circuit 11 and the like.

Thus, in the present embodiment, before and after the LED 5 emits the light of a light quantity (first quantity) for detection of the pulse wave, the LED 5 is configured to emit light of a smaller light emitting quantity (second light quantity) lower than that of the light for detection. In this way, pseudo disturbance light is generated such that the influence of the disturbance light is accurately removed from the pulse waveform having the influence of the disturbance light superimposed on the light emitted by the LED 5 for the detection. The above is detailed below.

As shown in FIG. 4, compared with the amplitude of the pulse wave signal (pulse wave amplitude) of a normal detection, the amplitude of the pulse wave signal having the influence of the incident of the disturbance light becomes larger (e.g., it may become several ten times larger in one example). Therefore, the pulse wave cannot be accurately detected based on the signal when the disturbance light is incident.

Thus, it may be required that a signal indicative of the pulse wave made only by the disturbance light while the signal while the LED 5 is off is removed from the signal indicative of the pulse wave made by the normal light emission by the LED 5.

Firstly, the DC component is exclusively discussed instead of a pulse wave amplitude (i.e., AC component) among the pulse wave signal indicative of the signal strength. When the LED 5 emits the light, the DC component due to the light emission by the LED 5 becomes large because the light may be reflected by the surface or may not be absorbed but reflected by the tissues of something other than the capillary artery. In contrast, the DC component due to only the disturbance light becomes equal to or less than one tenth of the DC component due to the light emission by the LED 5. In other words, the DC component due to only the disturbance light becomes very small, and as a result, the pulse wave signal due to only the disturbance light indicates a small value. Also, in general, under a measurement environment with a low signal value (e.g., a low light quantity), a pulse wave amplitude required for an accurate detection cannot be obtained because of the sensitivity property of the PD 9 that receives the signal value. In other words, the PD 9 has a blind range (e.g., sensitivity deterioration range) for the light detection, and therefore, the PD 9 may not output a signal corresponding to the light quantity if the light quantity is less than a certain level. As a result, it is difficult to obtain the signal made only by the disturbance light, and therefore, it is difficult to remove the signal made only by the disturbance light from the signal due to the normal light emission by the LED 5 as considered above.

Thus, in the present embodiment, in order to detect the disturbance light at a sensitivity equivalent to the detection of the pulse wave, pseudo disturbance light is generated by making the LED 5 emit the light of a certain light quantity in addition to the normal light emission by the LED 5. Here, the certain light quantity is equal to or less than a half of the light quantity at the normal light emission.

In other words, the light quantity of the LED 5 is determined at a level such that light having a light quantity that exceeds the blind range of the PD 9 can be received by the PD 9. Here, typically, even when the PD 9 receives the light having the light quantity within the blind range, a signal that corresponds to a change of the light quantity for the light received by the PD 9 cannot be obtained. In other words, the light quantity of the LED 5 is made to emit the light of a certain light quantity such that the signal value due to only the disturbance light (specifically, the value of the DC component of the signal) is made large, allowing the PD 9 to output the signal corresponding to the light quantity of the disturbance light. The light of the certain light quantity is applied to generate the pseudo disturbance light to obtain the signal. Then, the signal obtained by the emission of the pseudo disturbance light is removed from the signal obtained by the light emission of the normal light quantity (i.e., the signal of the pulse component having the disturbance light component superimposed thereon) to obtain the signal corresponding only to the pulse component.

It is noted that each of the light quantity for detection of the pulse wave and the light quantity for the pseudo disturbance light can be changed depending on the user as long as a ratio, which is equal to or less than a half, of the light quantity for detection of the pulse wave to the light quantity for the pseudo disturbance light is kept.

c) Next, a procedure of the process for calculating the pulse rate by the above program is described.

(1) Firstly, a main routine is described.

The process is performed at a sampling frequency of 16 Hz (sampling interval of 62.5 ms), and the LED 5 is made emit the light intermittently. The LED 5 may emit the light continuously in another embodiment.

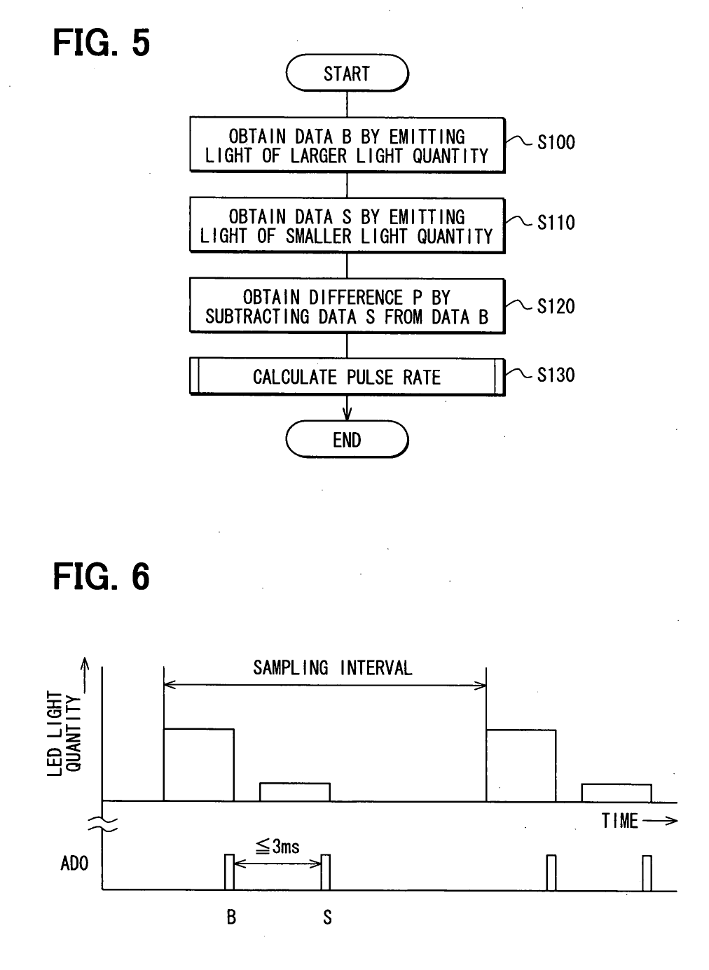

As shown in FIG. 5, at step S100, the light quantity of the light emitted by the LED 5 (i.e., light emitting quantity) is set to be large and equivalent to the light quantity of the light for the detection of the pulse wave at a normal condition. For example, the LED 5 emits the light for 1 ms, and the PD 9 receives the reflected light. Then, the detection circuit 11 detects the light receiving quantity for the reflected light, and data B (first signal) indicative of the light receiving quantity is obtained. For example, the obtaining of a signal in the present invention may indicate a process having steps of obtaining a signal outputted by a light receiving element, of reading the signal into a calculating device (e.g., a microcomputer) for an A/D conversion, and of storing the converted signal as data used for calculating a pulse rate.

Specifically, as shown in FIG. 6, data B (voltage signal) of the light receiving quantity during a light emitting interval a predetermined time (e.g., 0.1 ms) is stored synchronously with an end timing of the light emitting interval. It is noted that an upper positioned chart in FIG. 6 shows a light emitting timing, and a lower positioned chart in FIG. 6 shows an obtaining timing for the data (i.e., timing for obtaining the data used for calculating the pulse rate, and the like).

At step S110, after the LED 5 has stopped the above normal emission of the light, an interval of, for example, 0.5 ms is given. Then, the LED 5 emits the light of the smaller light emitting quantity for, for example, 1 ms to detect the disturbance light with a significant sensitivity. Then, the reflected light is received. Here, the smaller light emitting quantity corresponds to a half of the light emitting quantity (larger light emitting quantity) at the normal emission. Then, the detection circuit 11 detects the light receiving quantity for the reflected light to obtain data S (second signal).

Specifically, as shown in FIG. 6, the data S of the light receiving quantity during a next light emitting interval of a predetermined time (e.g., 0.1 ms) is stored synchronously with an end timing of the next light emitting interval. It is noted that an obtaining interval between (a) a timing for obtaining the data B at the first light reception and (b) a timing for obtaining the data S at the second light reception is set equal to or less than 3 ms, for example.

Here, a process order of step S100 and step S110 may be reversible. Also, the obtaining interval between a timing for obtaining the data B and a timing for obtaining the data S is set equal to or less than 3 ms as indicated above in order to accurately eliminate the disturbance light. A disturbance light quantity (i.e., the light quantity of the disturbance light) incident on the pulse wave sensor 1 always changes responsive to a physical relation between the pulse wave sensor 1 and the sun along with any environmental change or the physical movement of the user. Thus, when the light emitting interval becomes longer, an error of the disturbance light quantity included in each of the data B and the data S becomes larger. Therefore, when the obtaining interval is larger than 3 ms, the disturbance light may not be accurately eliminated.

At the following step S120, a difference P (=B−S ) is calculated by subtracting the data S of a case of the smaller light emitting quantity from the data B of a case of the larger light emitting quantity. Therefore, the data S that includes the disturbance light component is eliminated from the data B that includes the pulse component and the disturbance light component. As a result, the difference P that corresponds to the pulse component is selectively extracted.

A process of steps S100, S110, S120 is repeated at every sampling interval of 62.5 msec.

As shown in FIG. 6, the sampling interval is an interval from a start timing of the normal light emission of the light by the large light quantity by the LED 5 for detecting the pulse wave in the normal condition to a start timing of the next normal light emission by the LED 5. In the present embodiment, light is received twice to obtain two sets of data (data B, data S) of the different light quantities during the sampling interval. Then, the above data B and data S are stored.

At step S130, the pulse rate calculation process is executed using the data B, the data S, the difference P.

(2) Next, the pulse rate calculation process is described.

The pulse rate calculation process may employ various well-known calculation processes.

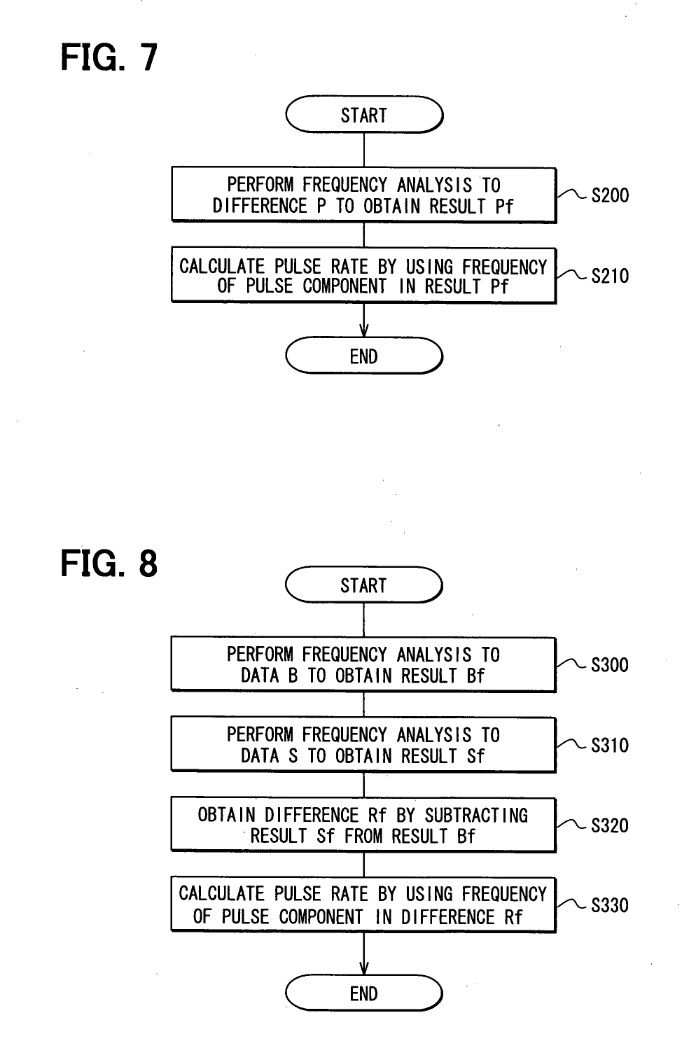

For example, the calculation process may employ a process for storing data at any time and then performing the frequency analysis of the data. Specifically, for example, as shown in FIG. 7, at step S200, a frequency analysis is performed to the difference P between data B and data S in order to obtain a frequency analysis result Pf. Here, the data B corresponds to a case of the larger light emitting quantity, and the data S corresponds to a case of the smaller light emitting quantity.

It is noted that the frequency analysis may employ, for example, fast Fourier transform (FFT) that is performed to time series information of each data. As a result, data indicating peaks in frequency as shown in the FIG. 3 can be obtained.

At step S210, the pulse rate is calculated by using a frequency at a largest peak of the pulse component of the frequency analysis result Pf obtained by the frequency analysis of the difference P.

Specifically, the pulse rate is calculated by multiplying (a) the frequency at the largest peak of the pulse component by (b) 60 seconds. For example, when the frequency is 1 [Hz], the pulse rate is calculated by the following equation of 1 [Hz]×60 [seconds]=60 [pulse/minute]. Also, a pulse interval is calculated by inverting the number of the above frequency.
Also, as an alternative method for calculating the pulse rate, for example, as shown in FIG. 8, at step S300, a frequency analysis is performed to the data B of the case of the larger light emitting quantity instead of using the above difference P in order to obtain a frequency analysis result Bf.

At step S310, a frequency analysis is performed to the data S of the case of the smaller light emitting quantity in order to obtain a frequency analysis result Sf.

At step S320, a difference Rf (=Bf−Sf ) of power spectrum of the frequency is obtained by subtracting the frequency analysis result Sf from the frequency analysis result Bf.

At step S330, the frequency at a largest peak of the pulse component of the difference Rf is used to calculate the pulse rate.

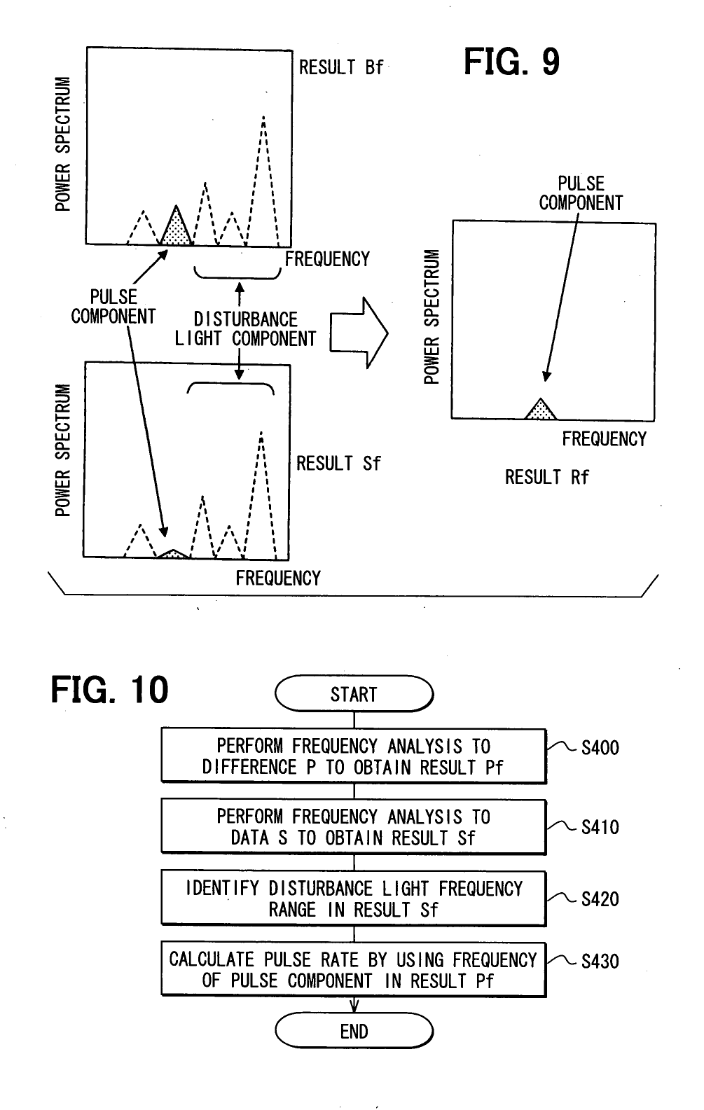

The result of the above process is shown in FIG. 9. The pulse component is hidden or not easily identified because of the disturbance light component when only the frequency analysis result Bf of the larger light emitting quantity is used. However, when the difference Rf between the frequency analysis result Sf and the frequency analysis result Bf is used, the pulse component can be exclusively obtained. Here, the frequency analysis result Sf is for the smaller light emitting quantity, and includes the disturbance light component equivalent to the case of the frequency analysis result Bf.
Also, in a case of outdoor exercise, such as walking, running, because a period of physical movement directly corresponds to a change period of the disturbance light (i.e., disturbance period), a process shown in FIG. 10 is executed. In the process, a frequency range corresponding to the period of the disturbance light is identified based on the frequency analysis result Sf of the smaller light emitting quantity, and the peak of the frequency other than the identified frequency range is detected.

Specifically, as shown in FIG. 10, at step S400, the difference P between the data B of the larger light emitting quantity and the data S of the smaller light emitting quantity is calculated, and the frequency analysis is performed to the difference P to obtain the frequency analysis result Pf.

At step S410, the frequency analysis is performed to the data S of the smaller light emitting quantity in order to obtain the frequency analysis result Sf.

At step S420, the frequency range of the disturbance light is identified based on the frequency analysis result Sf of the data S of the smaller light emitting quantity.

At step S430, a frequency at a largest peak other than the frequency range of the disturbance light in the frequency analysis Pf of the difference P is set as the frequency of the pulse component, and the pulse rate is calculated based on the frequency of the pulse component.

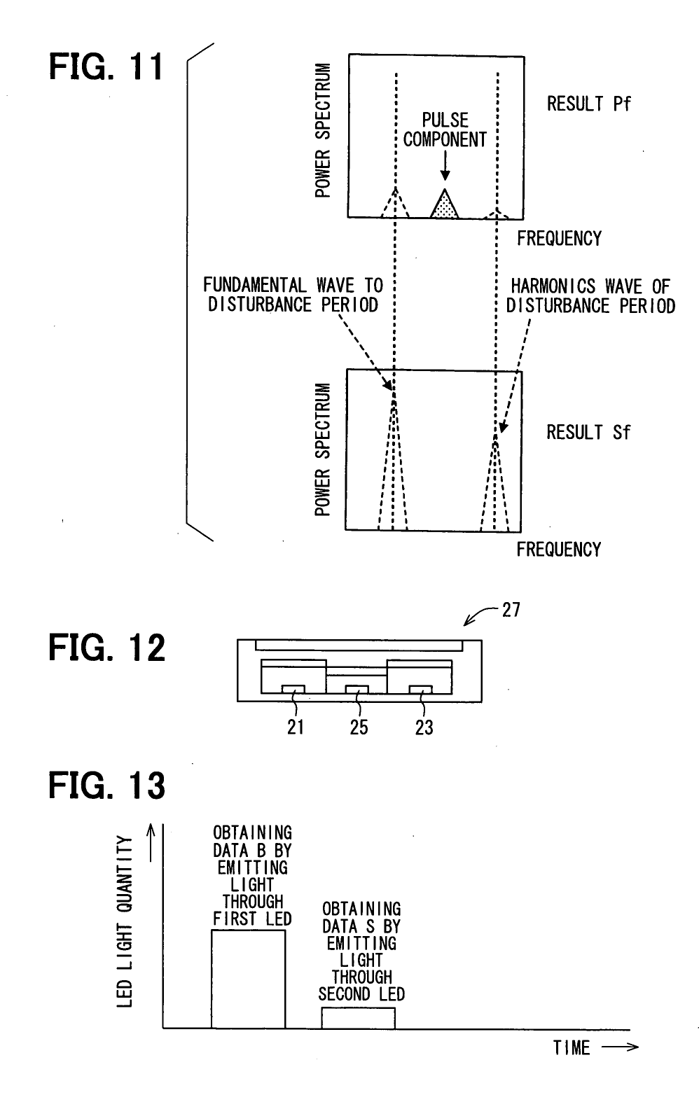

The result of the process is shown in FIG. 11. As indicated by the frequency analysis result Sf of the smaller light emitting quantity, the peaks of the frequency of a fundamental wave and a harmonics wave of the disturbance light are remarkable because the disturbance light changes at a certain period during the running. Thus, by using the above characteristic, even when the frequency analysis result Pf of the difference P between the data B and the data S is influenced by the physical movement of the user, the elimination of the influence of the disturbance light that changes with the certain period enables an accurate extraction of a frequency that corresponds to the pulse rate exclusively.

d) In the above way, in the present embodiment, the LED 5 emits the light of the large light emitting quantity for detection of the pulse wave, and the reflected light is received. Also, the light of the smaller light emitting quantity smaller than that of the light for detection of the pulse wave is applied as the pseudo disturbance light, and the reflected light is received. Then, because the above process for computing the pulse rate is performed, the pulse rate can be accurately detected even when the disturbance light exists. For example, the pulse rate is calculated based on the difference between the data sets corresponding to the respective light receiving quantities.

### Second Embodiment

Next, the second embodiment is described. Similar components similar to those in the first embodiment are indicated by the same numerals, and explanation thereof is omitted.

The present embodiment is slightly different from the first embodiment in a structure of a hardware.

As shown in FIG. 12, a pulse wave detection apparatus of the present embodiment employs a pulse wave sensor 27 that includes two LEDs (first LED 21, second LED 23) and a PD 25.

In the present embodiment, as shown in FIG. 13, the first LED 21 emits light of a larger light emitting quantity, and the second LED 23 emits light of a smaller light emitting quantity that is smaller than the larger light emitting quantity of the first LED 21.

In the present embodiment, similar advantages similar to the first embodiment can be achieved.

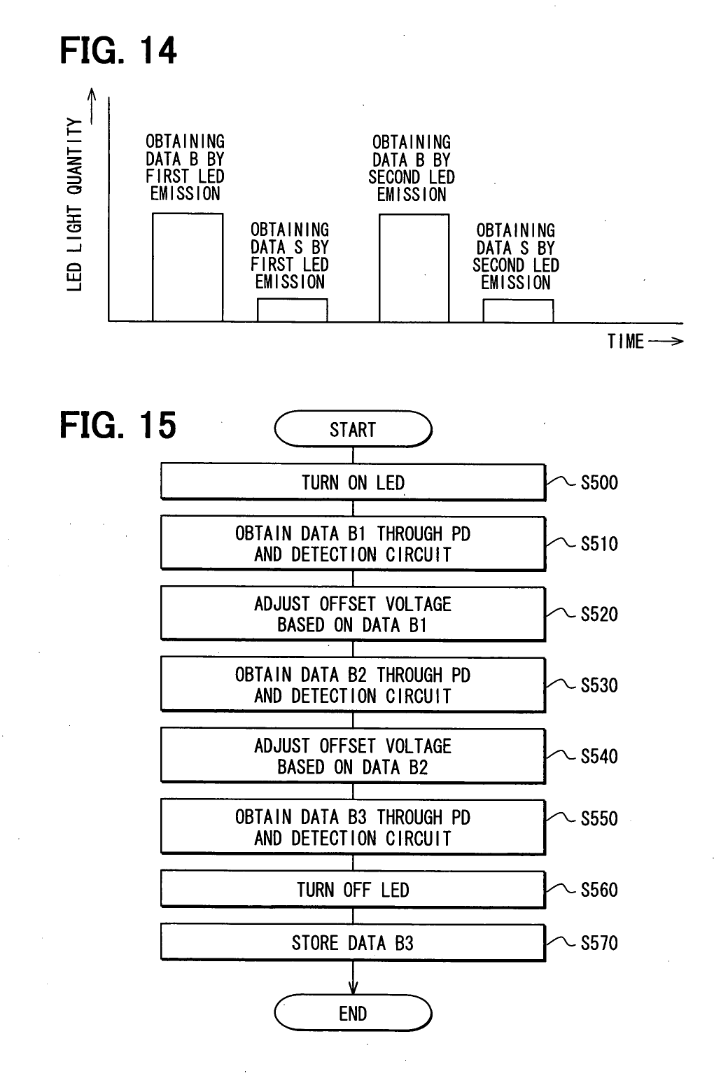

Also, as shown in FIG. 14, the light quantity of each of the two LEDs 21, 23 may be increased and decreased similarly to the first embodiment. However, in the present embodiment, the light quantity of each of the two LEDs 21, 23 is alternately changed so that after the first LED 21 emits the light of the larger light emitting quantity and the light of the smaller light quantity, and then, the second LED 23 emits the light of both light quantities. It is noted that the wave length of each of the LEDs 21, 23 may be different from each other or equal to each other.

### Third Embodiment

Next, the third embodiment is described. Similar components similar to those in the first embodiment are indicated by the same numerals, and explanation thereof is omitted.

The present embodiment is similar to the first embodiment in the structure of the hardware, but is different in a control process.

In the present embodiment, in order to detect single sampling data of the pulse wave, multiple data sets (two or more data sets) are obtained with the same light quantity (certain quantity) so that accuracy of the obtained data can be improved. It is noted that the single sampling data indicates representative data that is used in the frequency analysis to be followed.

Also, in the first embodiment, the data B and the data S are obtained by applying the light of the larger light emitting quantity and the light of the smaller light emitting quantity. The first embodiment is different from the present embodiment because the two lights having mutually different light emitting quantities are applied in order to obtain two sets of single sampling data in the first embodiment. It is noted that in a fourth embodiment described later, a combination of the first embodiment and the third embodiment will be described.

a) Firstly, a principle of the present embodiment is described.

Normally, in order to detect the single sampling data of the pulse wave, is needed to obtain the data only once. However, when the disturbance light is incident, the waveform of the pulse wave is widely disturbed. Thus, the amplitude of the pulse wave does not stay within the input voltage range (obtainable range), and the detectable amplitude is limited by the upper limit or the lower limit of the input voltage range. In other words, the amplitude of the input voltage ranges over the input voltage range of the pulse wave detection apparatus. Thus, when obtainment (sampling) of the data is repeated several times, the data may not be obtained accurately for each of the all obtaining processes depending on the sampling condition (e.g., disturbance light). This may greatly influence the pulse rate analysis.

Therefore, in the present embodiment, as a method for obtaining the data multiple times with the same light quantity, there is employed a process for optimizing a detection control and drive control based on previously obtained data before next data is obtained. In other words, in order to the single sampling data, the data is obtained multiple times.

Therefore, the single sampling data can be certainly detected without the limitation by the upper end or lower end the range. Then, the above process is executed for all samplings such that the accurate pulse waveform can be formed.

In the present embodiment, as described below, multiple-times obtainment of the data and offset voltage adjustment are repeated to accurately obtain the data.

It is noted that a known method described in, for example, JP-A-2005-160641 may be employed as a method for executing the offset voltage adjustment based on the previously detected data (preceding data). In other words, the offset voltage adjustment adjusts the direct-current component of signal received by the PD 9 (i.e., an offset voltage) based on the previously detected data. In this way, the pulse wave is detected in a condition that the amplitude of the pulse wave is not limited by the upper limit or the lower limit of the input voltage range.

b) Next, the process according to the present embodiment is described by referring to a flow chart in FIG. 15 and a timing chart in FIG. 17.

For example, as shown in FIG. 15 and FIG. 17, in order to detect the single sampling data, the three data sets (data B1, data B2, data B3) are obtained by equal intervals during a single light emission (light emitting interval).

It is noted that the interval between each data obtainment is equal to or less than 1 ms, and the obtainment of the third data synchronized with the end of the light emitting interval.

At step S500 in FIG. 15, firstly, the light of the light quantity for detection of the pulse wave is emitted.

At step S510, the reflected light is received by the PD 9, and at a first obtaining timing, first data B1 is obtained.

At step S520, the offset voltage is adjusted based on the first data B1.

At step S530, at a second obtaining timing, second data B2 is obtained.

At step S540, the offset voltage is again adjusted based on the second data B2.

At step S550, third data B3 is obtained at a third obtaining timing.

At step S560, the LED 5 is turned off.

At step S570, the third data B3 is stored as the representative data (single sampling data) used for the frequency analysis, and then the process is temporally ended.

c) In the above way, in the present embodiment, the data sets B1 to B3 are obtained. However, because the third data B3 that is lastly obtained is assumed to have a high accuracy, the third data B3 is employed as the single sampling data that is actually used for the calculation of the pulse rate. In other words, in the present embodiment, it is advantageous that the data has the high accuracy.

Also, in the present embodiment, because the data is obtained three times per the single light emission, it is not required to wait for the PD 9 and an operational amplifier (not shown) to be stable, and the interval between the obtainment of the data can be made shorter compared with, for example, the case shown in FIG. 16. FIG. 16 shows a case, where the light of the same light quantity is intermittently emitted three times, and the data is obtained at the timing of each light emission.
Alternatively, a leveling process for averaging multiple data sets may be employed instead of employing one obtained data as the single sampling data.
Also, specific data that stays within the input voltage range and that is accurately detected may be employed. In the method, although the sampling interval may be slightly different from each other for each obtainment of the data, unnecessary light emission by the LED 5 and the unnecessary obtainment of the data are not required. As a result, energy consumption can be reduced. In other words, it is not required that the data be obtained by the same number of times for each detection.
Alternatively, in contrast, immediately after the start of the measurement of the pulse wave, the number of times for obtaining the data may be. increased such that the responsiveness of the pulse rate detection can be improved. In this way, because the number of times for obtaining the data is increased when the measurement condition is not stable as above, advantages are achieved. Here, the unstable condition includes a case where the disturbance light is incident. In other words, the number of times for obtaining the data may be changeable with a condition of the sampling. Thus, the number of the plurality of signals obtained is changeable in accordance with a condition for detecting the single sampling data.

### Fourth Embodiment

Next, the fourth embodiment is described. Similar components similar to those in the first embodiment are indicated by the same numerals, and explanation thereof is omitted.

The present embodiment is similar to the first embodiment in the structure of the hardware, but is different from the first embodiment in a control process.

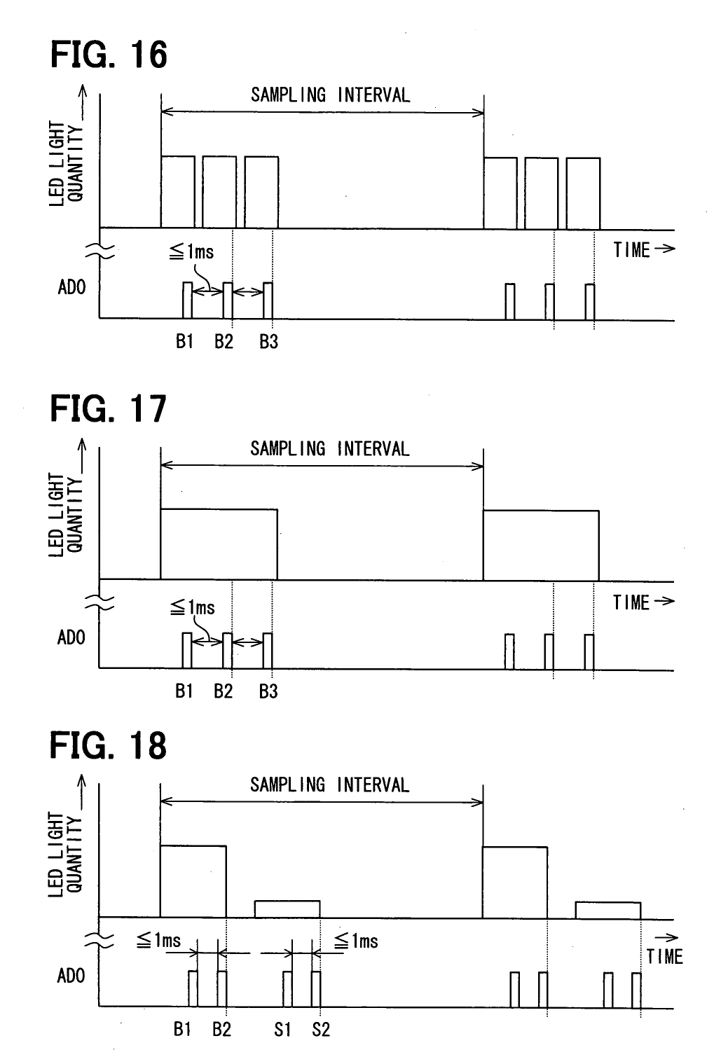

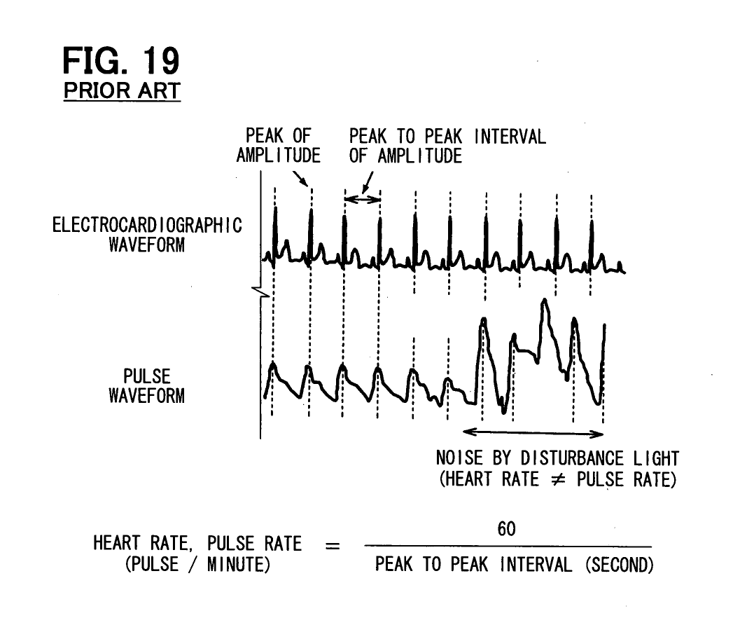

As shown in FIG. 18, in the present embodiment, the normal light of the larger light emitting quantity for detection of the pulse wave is emitted firstly, and then, the light of the smaller light emitting quantity smaller than that of the normal light is emitted for the pseudo disturbance light.

Then, when the light of the larger light emitting quantity is emitted for detection of the pulse wave, two data sets (data B1, data B2) are obtained at two obtaining timings. Also, when the light of the smaller light emitting quantity is emitted for the pseudo disturbance light, similarly, two data sets (data S1, data S2) are obtained at two obtaining timings.

The latter one (e.g., data B2) of the two sets of data (e.g., data B1, data B2) may be used for calculating the pulse rate, but an average of the two data sets may be used instead.

In this way, similar to the first embodiment, the influence from the disturbance light can be effectively eliminated, and also similar to the third embodiment, the obtained data is limited from ranging over the input voltage range. Thus, the highly accurate data can be advantageously obtained.

The present invention is not limited to the above embodiments, but can be applied to various embodiments.

(1) For example, programs for executing the above processes in the above embodiments are also within the scope of the present invention.

(2) Also, in the above embodiments, the pulse wave detection apparatus includes the pulse wave sensor. However, the pulse wave detection apparatus may be alternatively a device (e.g., data processing device) that executes a process for detecting the above pulse wave.

In the above embodiments, for example, the light quantity of the light emitting element (i.e., LED) is changeable with application of the electric current.

Additional advantages and modifications will readily occur to those skilled in the art. The invention in its broader terms is therefore not limited to the specific details, representative apparatus, and illustrative examples shown and described.

What is claimed is:

1. An optical pulse wave detection apparatus comprising:
a light emitting element that emits light to a living organism;
a light receiving element that receives a reflected light of the light that is reflected by the living organism;
a first control unit for obtaining a first signal by causing the light emitting element to emit the light of a first light quantity, and by causing the light receiving element to receive the reflected light of the light of the first light quantity;
a second control unit for obtaining a second signal by causing the light emitting element to emit the light of a second light quantity that is smaller than the first light quantity, and by causing the light receiving element to receive the reflected light of the light of the second light quantity; and
a pulse wave detection unit for detecting a pulse wave of the living organism based on the first signal and the second signal.

2. The pulse wave detection apparatus according to claim 1, wherein the second light quantity is equal to or less than a half of the first light quantity.

3. The pulse wave detection apparatus according to claim 1, wherein the second light quantity is set at a level such that the reflected light that is received by the light receiving element has a light quantity other than a sensitivity deterioration range of the light receiving element.

4. The pulse wave detection apparatus according to claim 1, wherein an interval between a first timing for obtaining the first signal and a second timing for obtaining the second signal is set equal to or less than 3 msec.

5. The pulse wave detection apparatus according to claim 1, wherein a ratio of the first light quantity to the second light quantity is kept when both the first light quantity and the second light quantity are changed in accordance with a condition.

6. The pulse wave detection apparatus according to claim 1, wherein the pulse wave is detected based on a difference between the first signal and the second signal.

7. The pulse wave detection apparatus according to claim 6, wherein the pulse wave is detected by performing a frequency analysis using the difference.

8. The pulse wave detection apparatus according to claim 1, wherein the pulse wave is detected based on a difference between a result of a first frequency analysis using the first signal and a result of a second frequency analysis using the second signal.

9. The pulse wave detection apparatus according to claim 1, wherein a disturbance period is specified based on a result of frequency analysis using the second signal.

10. The pulse wave detection apparatus according to claim 9, wherein the pulse wave is detected by comparing the disturbance period with a result of a frequency analysis using a difference between the first signal and the second signal.

11. An optical pulse wave detection apparatus comprising:
a light emitting element that emits light to a living organism;
a light receiving element that receives a reflected light of the light that is reflected by the living organism;
a signal control unit for causing the light emitting element to emit the light to the living organism, the signal control unit obtaining a plurality of signals from the light receiving element that receives the reflected light of the light emitted by light emitting element, each of the plurality of signals being obtained at a timing different from each other; and
a pulse wave detection unit for detecting single sampling data based on the plurality of signals, the single sampling data being used for detecting a pulse wave.

12. The pulse wave detection apparatus according to claim 11, wherein an interval between the timing for obtaining each of the plurality of signals is equal to or less than 1 msec.

13. The pulse wave detection apparatus according to claim 11, wherein:
the signal control unit causes the light emitting element to emit the light of a certain light quantity for a plurality of times; and
the signal control unit obtains the each of the plurality of signals at each of the plurality of times for causing the light emitting element to emit the light.

14. The pulse wave detection apparatus according to claim 11, wherein a next one of the plurality of signals is adjusted based on a preceding one of the plurality of signals, the preceding one preceding the next one.

15. The pulse wave detection apparatus according to claim 11, wherein a number of the plurality of signals obtained by the signal control unit is changeable in accordance with a condition for detecting the single sampling data.

16. The pulse wave detection apparatus according to claim 11, wherein a number of the plurality of signals is determined such that at least one of the plurality of signals has a magnitude that stays within an obtainable range of the signal control unit.

17. The pulse wave detection apparatus according to claim 11, wherein a certain one of the plurality of signals is set as the single sampling data, the certain one being obtained at a certain order among the plurality of signals.

18. The pulse wave detection apparatus according to claim 11, wherein:
each of the plurality of signals has a magnitude that stays within an obtainable range of the signal control unit; and
a first one or a last one of the plurality of signals is set as the single sampling data, the first one of the plurality of signals being obtained firstly, the last one of the plurality of signals being obtained lastly.

19. The pulse wave detection apparatus according to claim 11, wherein a calculation result using two or more of the plurality of signals is set as the single sampling data, each of the two or more of the plurality of signals having a magnitude that stays within an obtainable range of the signal control unit.

20. The pulse wave detection apparatus according to claim 11, further comprising:
the first control unit, the second control unit, and the pulse wave detection unit according to claim 1.

21. An article manufacture comprising:
a computer readable medium readable by a computer; and
program instructions carried by the computer readable medium for causing the computer to serve as the first control unit, the second control unit, and the pulse wave detection unit according to claim 1.

22. An article manufacture comprising:
a computer readable medium readable by a computer; and
program instructions carried by the computer readable medium for causing the computer to serve as the signal control unit and the pulse wave detection unit according to claim 11.
